(19) United States
(12) Patent Application Publication
(10) Pub. No.: US 2021/0332438 A1
(43) Pub. Date: Oct. 28, 2021

(54) Methods for predicting the risk of developing radio-induced cancer

(71) Applicants: UNIVERSITE DE BOURGOGNE, DIJON (FR); CENTRE GEORGES FRANCOIS LECLERC, DIJON (FR)

(72) Inventors: Philippe MAINGON, DIJON (FR); Céline MIRJOLET, MAGNY SUR TILLE (FR); Romain BOIDOT, ARCEAU (FR); Caroline TRUNTZER, DIJON (FR)

(21) Appl. No.: 16/625,319

(22) PCT Filed: Jun. 21, 2018
(86) PCT No.: PCT/EP2018/066679

§ 371 Date: Dec. 20, 2019

(30) Foreign Application Priority Data
Jun. 21, 2018 (FR) 17 55686

(51) Int. Cl. C12Q 1/6886

(52) U.S. Cl. CPC C12Q 1/6886; C12Q 2600/118; C12Q 2600/156

(57) ABSTRACT

The present invention relates to methods for predicting the risk of developing a sarcoma after radiotherapy treatment.

### TECHNICAL FIELD OF THE INVENTION

The present invention relates to methods for predicting the risk of developing a sarcoma after radiotherapy treatment.

### TECHNOLOGICAL BACKGROUND

Radiotherapy and surgery constitute the two major pillars of locoregional cancer treatment. More than 50% of cancer patients will be managed with radiotherapy. In 2015 this number represented 204 471 patients (INCa 2017 data), about a quarter of whom were treated for breast cancer. In France, 900 children are treated by radiotherapy each year.

The risk of appearance of a second cancer following radiotherapy treatment is being evaluated better and represents a major concern in terms of radioprotection. Among the malignant lesions discovered in treated patients, the appearance of a sarcoma is an event that is rare but of poor prognosis since the patient survival rate is estimated at between 10 to 35% at 5 years. The risk factors of this type of occurrence are unknown and the estimates of the incidence of this development vary from 1% to 1.75% in the surviving irradiated patients. On average, development of these sarcomas is observed between 5 and 10 years after radiotherapy.

Currently, the only studies conducted into the risk of developing radio-induced cancer have focused on dosimetry, investigating the doses received on the tissues where the cancer develops (Berrington de Gonzalez et al. 2012, Clinical Sarcoma Research, 2:18). However, no study has uncovered any genetic factors implicated in the risk of developing cancer induced by radiotherapy treatment.

There is therefore a real need to identify new factors for accurately evaluating the risks of a patient developing secondary cancer following radiotherapy treatment. Evaluation of this risk is essential for ensuring the best possible management for the patients, for example by avoiding radiotherapy treatment for them, if possible, or by providing them with close monitoring after the treatment when the risk associated with the appearance of a second cancer is high.

### DETAILED DESCRIPTION OF THE INVENTION

The present inventors have been able to demonstrate that the presence of nucleotide changes within the sequence of a specific group of genes made it possible to determine, with great selectivity and specificity, a patient's risk of developing radio-induced cancer. They demonstrated in particular that the detection of a nucleotide change within the sequence of at least 11 of the genes selected from the group consisting of KLRF1, C1orf234, PKD2L2, ZNF845, GRB2, TMEM117, KIRREL2, GCNT7, USP3, C1R, TOE1, QSER1, SCAMP1, SEPT7, SLC4A3, TBC1D10A, ZFP41, ZNF2, ICAM2, PRG4, MZT1, SYCE1, MAP1B, PRSS35, RPL11, JMJD4, SHISA4, FZD5, ISCU, LGI1, OSCAR, KDM4B, MIR3199-1, MIR3199-2, LIPI, ATP11C, ESF1, BCAS3, FAM63A, SNAP47, MUTYH, TTC28-AS1, RTFDC1 and SPRNP1, made it possible to predict the risk for a patient of developing radio-induced cancer.

Thus, according to a first aspect, the present invention relates to an in vitro method for determining the risk of developing radio-induced cancer in a patient treated by radiotherapy, said method comprising a step of detecting, in a biological sample obtained from said patient, a nucleotide change in the sequence of at least 11 genes selected from the group consisting of KLRF1, C1orf234, PKD2L2, ZNF845, GRB2, TMEM117, KIRREL2, GCNT7, USP3, C1R, TOE1, QSER1, SCAMP1, SEPT7, SLC4A3, TBC1D10A, ZFP41, ZNF2, ICAM2, PRG4, MZT1, SYCE1, MAP1B, PRSS35, RPL11, JMJD4, SHISA4, FZD5, ISCU, LGI1, OSCAR, KDM4B, MIR3199-1, MIR3199-2, LIPI, ATP11C, ESF1, BCAS3, FAM63A, SNAP47, MUTYH, TTC28-AS1, RTFDC1 and SPRNP1.

The inventors demonstrated in particular that the presence of a nucleotide change in the sequence of at least 11 of these genes was an indicator of increased risk of developing cancer following radiotherapy treatment.

Thus, in another aspect, the invention relates to an in vitro method for determining the risk of developing radio-induced cancer in a patient treated by radiotherapy, said method comprising a step of detecting, in a biological sample obtained from said patient, a nucleotide change in the sequence of at least 11 genes selected from the group consisting of KLRF1, C1orf234, PKD2L2, ZNF845, GRB2, TMEM117, KIRREL2, GCNT7, USP3, C1R, TOE1, QSER1, SCAMP1, SEPT7, SLC4A3, TBC1D10A, ZFP41, ZNF2, ICAM2, PRG4, MZT1, SYCE1, MAP1B, PRSS35, RPL11, JMJD4, SHISA4, FZD5, ISCU, LGI1, OSCAR, KDM4B, MIR3199-1, MIR3199-2, LIPI, ATP11C, ESF1, BCAS3, FAM63A, SNAP47, MUTYH, TTC28-AS1, RTFDC1 and SPRNP1, the presence of a nucleotide change in the sequence of at least 11 of these genes being synonymous with increased risk for the patient of developing a radio-induced sarcoma.

In another embodiment, for carrying out the method according to the present invention, a nucleotide change will be sought in 12 of the 44 genes stated above. In particular, the method according to the present invention may comprise the detection of nucleotide changes in 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43 or 44 of these genes. The method according to the present invention may also comprise the detection of a nucleotide change in all the 44 genes according to the present invention.

In the context of the present invention, the “biological sample” may be any sample in which human DNA can be detected. Typically, human DNA may be detected in lymphocytes derived from a blood sample, skin punch, or a saliva sample.

All the genes according to the invention are known as such and are listed in the following table:

TABLE A
List of the genes according to the invention

| Gene symbol | Name of the Gene | Gene ID |
|---|---|---|
| ESF1 | ESF1, nucleolar pre-rRNA processing protein, homolog | ENSG00000089048 |
| | (S. cerevisiae) | |
| PRSS35 | protease, serine, 35 | ENSG00000146250 |
| TMEM117 | transmembrane protein 117 | ENSG00000139173 |
| QSER1 | glutamine and serine rich 1 | ENSG00000060749 |
| SYCE1 | synaptonemal complex central element protein 1 | ENSG00000171772 |
| ZNF2 | zinc finger protein 2 | ENSG00000163067 |
| LIPI | lipase, member I | ENSG00000188992 |
| LGI1 | leucine-rich, glioma inactivated 1 | ENSG00000108231 |
| OSCAR | osteoclast associated, immunoglobulin-like receptor | ENSG00000170909 |
| MIR3199-1 | microRNA 3199-1 | ENSG00000264073 |
| SPRNP1 | shadow of prion protein homolog (zebrafish) pseudogene 1 | NA |
| SLC4A3 | solute carrier family 4, anion exchanger, member 3 | ENSG00000114923 |
| JMJD4 | jumonji domain containing 4 | ENSG00000081692 |
| KDM4B | lysine (K)-specific demethylase 4B | ENSG00000127663 |
| TOE1 | target of EGR1, member 1 (nuclear) | ENSG00000132773 |
| ICAM2 | intercellular adhesion molecule 2 | ENSG00000108622 |
| SNAP47 | synaptosomal-associated protein, 47 kDa | ENSG00000143740 |
| GCNT7 | glucosaminyl (N-acetyl) transferase family member 7 | ENSG00000124091 |
| MIR3199-2 | microRNA 3199-2 | ENSG00000283225 |
| ISCU | iron-sulfur cluster scaffold homolog (E. coli) | ENSG00000136003 |
| USP3 | ubiquitin specific peptidase 3 | ENSG00000140455 |
| MAP1B | microtubule-associated protein 1B | ENSG00000131711 |
| MUTYH | mutY homolog (E. coli) | ENSG00000132781 |
| SCAMP1 | secretory carrier membrane protein 1 | ENSG00000085365 |
| KIRREL2 | kin of IRRE like 2 (Drosophila) | ENSG00000126259 |
| MZT1 | mitotic spindle organizing protein 1 | ENSG00000204899 |
| RPL11 | ribosomal protein L11 | ENSG00000142676 |
| FZD5 | frizzled family receptor 5 | ENSG00000163251 |
| C1R | complement component 1, r subcomponent | ENSG00000159403 |
| PKD2L2 | polycystic kidney disease 2-like 2 | ENSG00000078795 |
| TTC28-AS1 | TTC28 antisense RNA 1 | ENSG00000235954 |
| ZNF845 | zinc finger protein 845 | ENSG00000213799 |
| TBC1D10A | TBC1 domain family, member 10A | ENSG00000099992 |
| SHISA4 | shisa homolog 4 (Xenopus laevis) | ENSG00000198892 |
| ATP11C | ATPase, class VI, type 11C | ENSG00000101974 |
| KLRF1 | killer cell lectin-like receptor subfamily F, member 1 | ENSG00000150045 |
| GRB2 | growth factor receptor-bound protein 2 | ENSG00000177885 |
| RTFDC1 | Replication Termination Factor 2 Domain Containing 1 | ENSG00000022277 |
| ZFP41 | Zinc Finger Protein | ENSG00000181638 |
| C1orf234 | Chromosome 1 Open Reading Frame 234 | ENSG00000227868 |
| PRG4 | Proteoglycan 4 | ENSG00000116690 |
| SEPT7 | Septin 7 | ENSG00000122545 |
| BCAS3 | breast carcinoma amplified sequence 3 | ENSG00000141376 |
| FAM63A | family with sequence similarity 63, member A | ENSG00000143409 |

A “nucleotide change” corresponds to a change of at least one nucleotide in a gene sequence. A change of at least one nucleotide may be:
a deletion, i.e. removal of at least one nucleotide in the gene sequence;
an insertion, i.e. addition of at least one nucleotide in the gene sequence;
a substitution, i.e. replacement of one nucleotide with another.

A person skilled in the art knows a great many techniques allowing him to detect a change of nucleotides within a gene.

A nucleotide change is typically detected in a patient by comparing the sequence of a nucleic acid expressed by said patient with the sequence of the corresponding nucleic acid expressed in a control population.

According to the present invention, the nucleotide change may be detected by analyzing the nucleic acid molecules of the genes according to the invention. These nucleic acid molecules are double-stranded genomic DNA.

As stated above, the nucleic acid molecule is obtained from any biological sample containing DNA, for example a blood sample. The DNA may be extracted by the methods described in the manual of Sambrook et al. (1989). A great many techniques may be used for analyzing a genotype (see for example Antonarakis et al., 1989; Cooper et al., 1991; Grompe, 1993). For example, we may mention analysis of restriction fragment length polymorphism (RFLP); hybridization with allele-specific oligonucleotides, which are small synthetic probes that only hybridize when there is perfect sequence identity in suitable hybridization conditions; allele-specific PCR, PCR with mutagenic primers; allelic discrimination, analysis of fragments, PCR HRM, ligase-PCR; denaturing gradient gel electrophoresis (DGGE); single-stranded conformational polymorphism (SSCP) and high-performance liquid chromatography on denaturing gel (Kuklin et al., 1997).

Direct sequencing may be carried out by any method known by a person skilled in the art, for example such as chemical sequencing by the method of Maxam and Gilbert; enzymatic sequencing by Sanger's method; sequencing by mass spectrometry; and next generation sequencing, regardless of the technology employed for sequencing (sequencing on solid support, nanopore sequencing, pH-metry sequencing, etc.) or for preparing the library (capture or amplicons).

Thus, nucleic acid molecules useful for the present invention are for example probes or primers. These probes and primers may in particular hybridize to the regions where the mutation is located. The nucleotide probes or primers may at least contain 10, 15, 20 or 30 nucleotides. Their length may be less than 400, 300, 200 or 100 nucleotides.

The nucleotide changes according to the present invention may be located in the exons (i.e. in the coding parts of the gene), but also in the introns (i.e. the noncoding regions of the gene). Thus, these nucleotide changes may have an effect on the protein encoded by the gene (missense mutations, nonsense mutations), or may have no effect on the protein sequence (silent mutations).

In a particular embodiment, the nucleotide change according to the present invention is found in less than 10% of the general population, in particular less than 5% of the general population, and preferably in less than 1% of the general population.

Typically the frame of reference “general population” uses all the available databases (example: 1000genomes, dbSNP, Ensembl, Exac, etc.).

In the context of the present invention, the “primary cancer”, i.e. the cancer for which the patient is treated by radiotherapy, is any cancer that can be treated with this type of therapy. In particular, this primary cancer is selected from the group consisting of breast cancer, cancer of the upper respiratory/digestive tract (ORL cancer), colorectal cancer, lung cancer, cervical cancer, endometrial cancer, ovarian cancer, prostate cancer, testicular cancer, brain tumors or tumors of the central nervous system, thyroid cancer and ophthalmic cancer. In a particular embodiment, said primary cancer is breast cancer.

The term “radio-induced cancer” according to the present invention signifies any cancer induced following radiotherapy treatment. In a particular embodiment, the cancer is a sarcoma, in particular a soft tissue sarcoma. “Radiotherapy” is a locoregional cancer treatment consisting of using radiation to destroy cancer cells. On average, patients treated with radiotherapy receive a radiation dose from 50 to 80 gray (Gy). In a particular embodiment, the method according to the invention is carried out before the patient is treated with radiotherapy. According to this embodiment, it is therefore possible to determine before the treatment whether the patient has a high risk of developing radio-induced cancer following the treatment. This notably allows the treatment strategy for this patient to be changed, when this is possible. For example, we may choose to treat this patient by surgery, by chemotherapy or by immunotherapy rather than with radiotherapy.

Thus, in another embodiment, the present invention also makes it possible to devise a method for treating a cancer patient, in which a patient said method comprising the following steps:
a) detecting, in a biological sample obtained from said patient, a nucleotide change in the sequence of at least 11 genes selected from the group consisting of KLRF1, C1orf234, PKD2L2, ZNF845, GRB2, TMEM117, KIRREL2, GCNT7, USP3, C1R, TOE1, QSER1, SCAMP1, SEPT7, SLC4A3, TBC1D10A, ZFP41, ZNF2, ICAM2, PRG4, MZT1, SYCE1, MAP1B, PRSS35, RPL11, JMJD4, SHISA4, FZD5, ISCU, LGI1, OSCAR, KDM4B, MIR3199-1, MIR3199-2, LIPI, ATP11C, ESF1, BCAS3, FAM63A, SNAP47, MUTYH, TTC28-AS1, RTFDC1 and SPRNP1.
b) based on the results obtained in step a), determining the risk of developing radio-induced cancer in said patient;
c) treating the patient by a treatment other than radiotherapy when the risk determined in step b) is increased.

It is also possible that the patient might not be treatable by a treatment other than radiotherapy. In this context, if the method according to the present invention shows an increased risk of the patient developing radio-induced cancer, it may be decided that the patient will have to be monitored more closely so as to be able to detect the appearance of radio-induced cancer as quickly as possible. Patients generally develop radio-induced cancer in 15 years, or 10 years and more particularly in 5 to 10 years following radiotherapy treatment. Thus, when a patient who has been treated or who will be treated by radiotherapy has a risk of developing radio-induced cancer according to the present invention, said patient will benefit from close monitoring during the 5, 10 or 15 years following the treatment. This follow-up will consist for example of monitoring for the appearance of cancer, for example by regularly measuring biomarkers representative of a given cancer. For example, it will be possible to monitor for the biomarkers that make it possible to detect and monitor the development of sarcomas. These markers are described for example in Satelli et al. (Cancer Res., 2014, 74(6), 1645-1650) or in Namlos et al. (BMC Cancer, 2017, 17(1), 29). The method according to the invention therefore makes it possible to detect the appearance of radio-induced cancer as early as possible and therefore instigate early management of said cancer, which greatly increases the patient's chances of survival. In this embodiment, the method according to the present invention may be carried out before or after treatment of the patient by radiotherapy.

According to the present invention, a “patient” is a human being. In a particular embodiment, the patient is a patient under the age of 18 years. In fact, as stated above, about 900 children are treated with radiotherapy each year in France. After treatment of their primary cancer, these children have a long life ahead of them. The effect of the treatment on the duration of life of these children in the long term is therefore an extremely important question. The use of the method according to the invention is therefore particularly advantageous in patients of this type.

The present invention is presented in more detail in the following examples. These examples are supplied purely for purposes of illustration and should not be interpreted as limiting the scope of the present invention.

### EXAMPLES

### Material and Methods:

We shall now demonstrate that detection of a nucleotide change in at least 11 of the genes presented in Table A allows accurate determination of a patient's risk of developing radio-induced cancer following radiotherapy treatment. The results demonstrate that detection of nucleotide changes in these genes makes it possible to predict radio-induced cancer in more than 80% of patients.

Patients and Samples:

The set of results that made it possible to generate this signature is derived from the SARI study (SARI: SArcoma Radio-Induced; study of the parameters predictive of the appearance of sarcomas that have developed in an irradiated area). The primary objective of this study was to find a genetic signature that could predict a patient's predisposition to develop a second cancer of the sarcoma type after being treated with radiotherapy for a first cancer. This case-control study required the inclusion of 360 patients: 120 CASE patients who have developed a radio-induced sarcoma and 240 CONTROL patients who received radiotherapy more than 5 years previously and have not developed a secondary sarcoma. The CASES/CONTROLS were paired on the basis of several criteria: patient's age at the time of the initial radiotherapy, sex, and the location of the primary tumor.

An 8-ml blood sample was taken for all of the patients in EDTA tubes. The lymphocytes were recovered by a FICOL technique and then the DNA was extracted.

The exomes were then prepared with the SureSelect XT HUMAN All Exon v5 kit (Agilent) and sequenced on a NextSeq500 sequencer (Illumina). The data generated were pretreated by an Illumina/Annotations Variant Studio process (depth of reading>10, frequency of the alternative allele>30, PASS Filter). The variants were then subdivided into two categories: Synonymous Variants and Intron/Coding Variants.

Multivariate statistical analysis was undertaken. Various methods for large-scale selection of variables were used (Sparse PLS, lasso, random forests). The set of variables selected by these different approaches was adopted and made it possible to select 44 variables*. A method of selecting variables based on random forests in a large-scale situation was then used on this subset of variables, with the aim of keeping a limited number of them. This method made it possible to identify the 44 genes in Table A, changes of which correlate with the patients' predisposition to develop a radio-induced sarcoma.

1-7. (canceled)

8. An in vitro method for treating a patient suffering or at risk of suffering from radio-induced cancer, wherein said method comprises the steps of:
i) detecting, in a biological sample obtained from said patient, a nucleotide change in the sequence of at least 11 genes selected from the group consisting of KLRF1, C1orf234, PKD2L2, ZNF845, GRB2, TMEM117, KIRREL2, GCNT7, USP3, C1R, TOE1, QSER1, SCAMP1, SEPT7, SLC4A3, TBC1D10A, ZFP41, ZNF2, ICAM2, PRG4, MZT1, SYCE1, MAP1B, PRSS35, RPL11, JMJD4, SHISA4, FZD5, ISCU, LGI1, OSCAR, KDM4B, MIR3199-1, MIR3199-2, LIPI, ATP11C, ESF1, BCAS3, FAM63A, SNAP47, MUTYH, TTC28-AS1, RTFDC1 and SPRNP1;
ii) determining, in view of the results obtained in step i), whether the patient suffers from or is at risk of developing a radio-induced cancer; and
iii) treating the patient accordingly.

9. The method according to claim 8, wherein the presence of a nucleotide change in the sequence of at least 11 genes among KLRF1, C1orf234, PKD2L2, ZNF845, GRB2, TMEM117, KIRREL2, GCNT7, USP3, C1R, TOE1, QSER1, SCAMP1, SEPT7, SLC4A3, TBC1D10A, ZFP41, ZNF2, ICAM2, PRG4, MZT1, SYCE1, MAP1B, PRSS35, RPL11, JMJD4, SHISA4, FZD5, ISCU, LGI1, OSCAR, KDM4B, MIR3199-1, MIR3199-2, LIPI, ATP11C, ESF1, BCAS3, FAM63A, SNAP47, MUTYH, TTC28-AS1, RTFDC1 and SPRNP1 indicates that the patient suffers from or is at risk of developing a radio-induced sarcoma.

10. The method according to claim 8, wherein the biological sample is a blood sample.

11. The method according to claim 8, wherein said nucleotide change is present in less than 1% of the general population.

12. The method according to claim 8, wherein the sample is obtained before treatment of said patient by radiotherapy.

13. The method according to claim 8, wherein the sample is obtained after treatment of said patient by radiotherapy.

14. The method according to claim 8, wherein the sample is obtained from a patient under 18 years of age.
